(19) United States
(12) Patent Application Publication
(10) Pub. No.: US 2026/0263023 A1
(43) Pub. Date: Sep. 10, 2026

(54) MOTION DETECTION USING HIGH CONTRAST FEATURES

(71) Applicant: Hologic, Inc., Marlborough, MA (US)

(72) Inventor: Ashwini KSHIRSAGAR, Cupertino, CA (US)

(73) Assignee: Hologic, Inc., Marlborough, MA (US)

(21) Appl. No.: 19/166,207

(22) PCT Filed: Mar. 22, 2024
(86) PCT No.: PCT/US2024/021212

§ 371 Date: Sep. 17, 2025

Related U.S. Application Data
(60) Provisional application No. 63/491,769, filed on Mar. 23, 2023.

(51) Int. Cl. A61B 6/00; A61B 6/50; G06T 7/00

(52) U.S. Cl. CPC A61B 6/5264; A61B 6/502; G06T 7/0012; G06T 2207/30068

(57) ABSTRACT

A method of detecting motion of a patient's breast tissue during medical imaging, including obtaining image data of the patient's breast tissue; processing the image data to generate a set of image slices that collectively depict the patient's breast tissue; detecting a high contrast object on one or more image slices of the set of image slices; identifying an artifact, associated with the high contrast object, on one or more image slices of the set of image slices; measuring a physical attribute of the artifact; measuring a deviation of the physical attribute from a baseline; determining the deviation exceeds a predetermined threshold; and generating, in response to determining the deviation exceeds the predetermined threshold, a motion indicator.

### CROSS-REFERENCE TO RELATED APPLICATIONS

This application is being filed as a PCT International Patent Application and claims priority to U.S. Provisional Patent Application No. 63/491,769, filed Mar. 23, 2023, entitled “MOTION DETECTION USING HIGH CONTRAST FEATURES,” which is incorporated herein by reference in its entirety.

### BACKGROUND

A major challenged faced in medical imaging is ensuring usability of the images produced. Patient movement during the course of an imaging sequence, for instance due to the length of the sequence or discomfort due to the arrangement of the imaging device, is a frequent source of distortions and artifacts which may render an image unusable. Distorted images often fail to accurately depict diagnostically relevant structures.

### SUMMARY

Examples presented herein are directed to a method of detecting motion of a patient's breast tissue during medical imaging, including obtaining image data of the patient's breast tissue; processing the image data to generate a set of image slices that collectively depict the patient's breast tissue; detecting a high contrast object on one or more image slices of the set of image slices; identifying an artifact, associated with the high contrast object, on one or more image slices of the set of image slices; measuring a physical attribute of the artifact; measuring a deviation of the physical attribute from a baseline; determining the deviation exceeds a predetermined threshold; and generating, in response to determining the deviation exceeds the predetermined threshold, a motion indicator.

In other examples presented herein, the baseline is a straight line. In further examples, the baseline includes a baseline range. In further examples, the baseline range is determined according to a set of physical attributes associated with one or more artifacts. In further examples, the one or more artifacts are associated with no patient movement. In further examples, the predetermined threshold is determined according to the baseline range. In further examples, the baseline range is measured using an R-square analysis.

In other examples presented herein, the high contrast object is identified on an in-focus image slice and the artifact is a projection of the high contrast object identified on an out-of-focus image slice. In other examples, detecting a high contrast object comprises identifying at least one high contrast object in a first image slice and, in response, scanning a second image slice one or more slices away from the first image slice for the artifact.

In other examples, the physical attribute includes one or more of a width, a length, a height, a trajectory, and an opacity. In further examples, determining the deviation exceeds a predetermined threshold is based on measuring the physical attribute associated with the artifact and measuring the at least one high contrast object.

In other examples presented herein, measuring the deviation from the baseline comprises applying a fitting algorithm, wherein inputs to the fitting algorithm comprise one or more of a size of the artifact, a size of the associated high contrast object, and a contrast of the artifact. In other examples, the high contrast object is a naturally occurring object in the breast including one or more of a calcification or a ligament. In other examples, the high contrast object is an implanted object. In other examples, the motion indicator comprises an indicator on at least one image of the one or more image slices. In other examples, the motion indicator comprises a request for immediate review of at least one image of the one or more images. In other examples, measuring the deviation from the baseline associated with the artifact comprises using an R-square analysis.

Other examples presented herein are directed to a system including a computer-readable memory storing executable instructions; and one or more processors in communication with the computer-readable memory, wherein, when the one or more processors execute the executable instructions, the one or more processors perform obtaining image data of the patient's breast tissue; processing the image data to generate a set of image slices that collectively depict the patient's breast tissue; detecting a high contrast object on one or more image slices of the set of image slices; identifying an artifact, associated with the high contrast object, on one or more image slices of the set of image slices; measuring a physical attribute of the artifact; measuring a deviation of the physical attribute from a baseline; determining the deviation exceeds a predetermined threshold; and generating, in response to determining the deviation exceeds the predetermined threshold, a motion indicator.

Other examples presented herein are directed to a non-transitory computer readable medium having stored thereon one or more sequences of instructions for causing one or more processors to perform: obtaining image data of the patient's breast tissue; processing the image data to generate a set of image slices that collectively depict the patient's breast tissue; detecting a high contrast object on one or more image slices of the set of image slices; identifying an artifact, associated with the high contrast object, on one or more image slices of the set of image slices; measuring a physical attribute of the artifact; measuring a deviation of the physical attribute from a baseline; determining the deviation exceeds a predetermined threshold; and generating, in response to determining the deviation exceeds the predetermined threshold, a motion indicator.

Examples presented herein are directed to a method of detecting motion of a patient's breast tissue during medical imaging, including receiving image data of the patient's breast tissue; processing the image data to generate a set of image slices that collectively depict the patient's breast tissue; detecting a high contrast object on one or more image slices of the set of image slices; identifying an artifact, associated with the high contrast object, on one or more image slices of the set of image slices; measuring a physical attribute of the artifact; measuring a deviation of the physical attribute from a baseline; determining the deviation exceeds a predetermined threshold; and generating, in response to determining the deviation exceeds the predetermined threshold, a motion indicator.

A variety of additional inventive aspects will be set forth in the description that follows. The inventive aspects can relate to individual features and to combinations of features. It is to be understood that both the forgoing general description and the following detailed description are exemplary and explanatory only and are not restrictive of the broad inventive concepts upon which the examples disclosed herein are based.

### BRIEF DESCRIPTION OF THE DRAWINGS

The accompanying drawings, which are incorporated in and constitute a part of the description, illustrate several aspects of the present disclosure. A brief description of the drawings is as follows:

FIG. 1A is a schematic view of an exemplary imaging system.

FIG. 1B is a perspective view of the imaging system of FIG. 1A.

FIG. 2 illustrates an example baseline of an artifact and an example deviation of an artifact.

FIG. 3 is an example of an artifact in an image.

FIG. 4 is an example of an artifact in an image obtained during patient motion.

FIG. 5 is an example workflow for detecting patient motion based on image artifacts.

FIG. 6 depicts an example of a suitable operating environment in which one or more of the present examples can be implemented.

### DETAILED DESCRIPTION

Described herein are systems and methods for detection and identification of images impacted by patient motion. Medical imaging frequently requires patients to maintain uncomfortable positions for a period of time to ensure the imaging device is properly arranged to have an unobstructed view of internal body structures. The combination of patient discomfort and the time required for an image capture sequence often results in the patient moving during the imaging process. Such movement may distort the structures within the image, thus producing images without diagnostic utility or relevance. Such images can result in a radiologist or other clinician being unable to timely identify a cancerous or otherwise significant structure of interest and, further, can lead to increased cost and inconvenience as the images must be recaptured. Aspects of the present disclosure may be particularly applicable to imaging modalities performed over a range of motion measured by a projection angle, such as tomosynthesis.

In x-ray-based medical imaging, such as of a breast, the density of various structures within the tissue of the breast produce image objects with differing contrast against a relatively low density background of the overall volume of breast tissue. Objects with a high relative density, such as calcifications, implanted clips or wires, or other structures, appear as high contrast objects. High contrast objects generally appear as relatively sharp or bright white objects against a darker background of the overall breast tissue. As used herein, an object refers to shapes or structures appearing in focus in a particular image, or image slice, in a set or stack of images that together depict an overall volume of breast tissue. The object is generally considered to be a true depiction of a structure in breast tissue.

In imaging modalities defined by a projection angle, e.g., less than 180 degrees, these high contrast objects can cast shadows which may appear in reconstructed images as artifacts tracking the plane of travel of the x-ray source. As used herein, an artifact refers to an effect created by the imaging system and not a true depiction of a structure in the breast. An artifact is generally associated with one object. An object may be associated with one or more artifacts. The term artifact may encompass any feature appearing in an image, but which is not present in the original imaged object. In examples, an artifact may be an object appearing out of focus in a particular image or image slice or a shadow cast by an object on a particular image slice due to the geography of the breast or the projection angle of the imaging system.

An artifact will generally track the movement of the x-ray source and appear with a straight line characteristic in the direct of movement of the x-ray tube. As used herein, a baseline refers to this straight line characteristic associated with artifacts showing movement of the x-ray tube. The baseline may encompass a set of characteristics including, by example, a center point, a width, a measurement in one or more directions beginning from the center point, etc.

The inventors on this application have identified an innovative insight that these artifacts may also track, and be visibly distorted by, motion of the patient during the image capture sequence. Therefore, the systems and methods disclosed herein enable these artifact distortions to be leveraged as motion indicators. By training or programming a system to identify artifact deviations as associated with patient movement, distorted images can be identified quickly and evaluated for usability, potentially saving the patient, and imaging team, the cost, time, and inconvenience associated with arranging another appointment to recapture images of the breast. As used here, a deviation refers to one or more characteristics of an artifact that differ from a set of baseline characteristics, e.g., a set of characteristics defining a straight line.

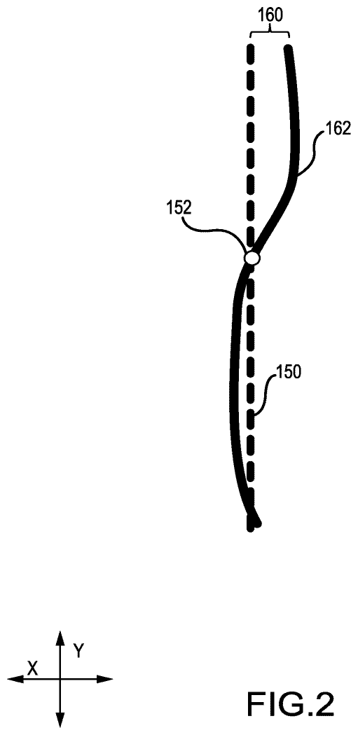

FIG. 2 illustrates an example baseline 150 of an artifact and an example deviation 160 of an artifact 160. Example baseline 150 appears as straight line in the direction of travel of the x-ray tube (the y-axis direction in FIG. 2). Baseline 150 may include a center point 152. Baseline 150 may be identified as a baseline by extending from center point 152 only along the y-axis or in the direction of travel of the x-ray source.

Example artifact 162 shows an example deviation 160 from baseline 150. In addition to extending away from center point 152 in the direction of movement of the x-ray source (the y-axis in FIG. 2), artifact 162 also deviates and extends away from center point 152 in a direction perpendicular to the travel of the x-ray source (the x-axis in FIG. 2). This additional movement is captured by deviation 160 and is not attributable to the movement of the x-ray source. Deviation 160 can be attributed to movement of a patient or other subject being imaged.

Other motion detection systems for tracking motion typically determine the location and distance to an anatomical structure, such as distance to the nipple or pectoral muscle and whether that distance have changed. Unlike the high contrast objects described herein, such anatomical structures are not typically high contrast. In yet other systems, motion may be detected using artificial markers positioned on an exterior skin line or on a portion of an imaging system such as a compression paddle. Instead, the current high contrast objects are located inside the breast and are either naturally occurring or placed inside the breast as a result of an interventional procedure. In addition, the systems and methods described herein determine not position of the high contrast object itself and instead use the appearance of the artifact that is produced by the high contrast object in the resulting image.

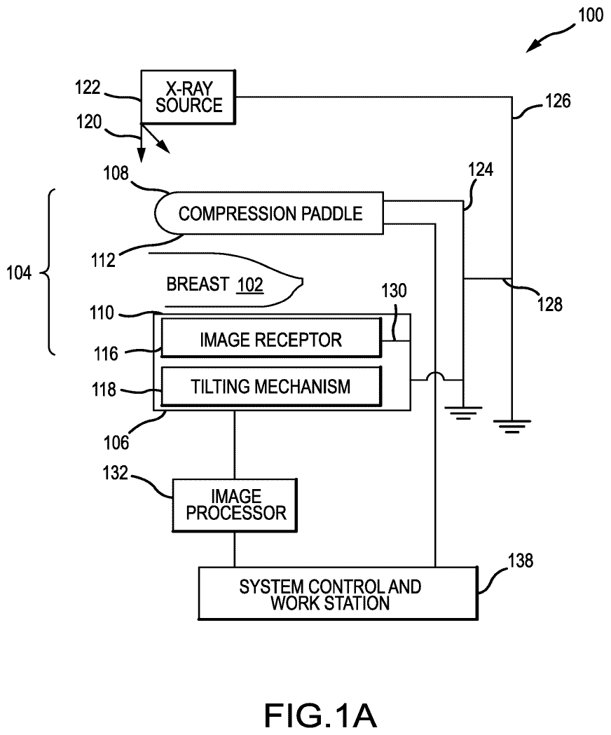

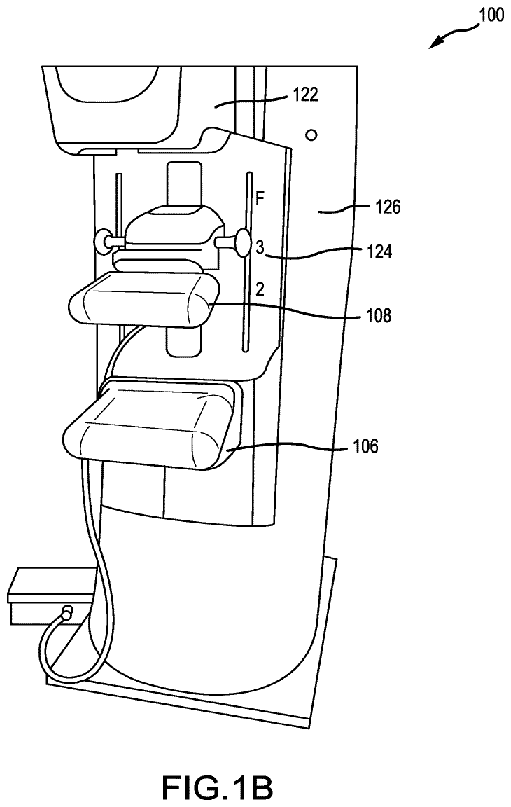

FIG. 1A is a schematic view of an exemplary imaging system 100 that produces the artifacts described in relation to FIG. 2. FIG. 1B is a perspective view of the imaging system 100. Referring concurrently to FIGS. 1A and 1B, the imaging system 100 immobilizes a patient's breast 102 for x-ray imaging (either or both of mammography and tomosynthesis) via a breast compression immobilizer unit 104 that includes a static breast support platform 106 and a moveable compression paddle 108. The breast support platform 106 and the compression paddle 108 each have a compression surface 110 and 112, respectively, that move towards each other to compress and immobilize the breast 102. In known systems, the compression surface 110, 112 is exposed so as to directly contact the breast 102. The platform 106 also houses an image receptor 116 and, optionally, a tilting mechanism 118, and optionally an anti-scatter grid. The immobilizer unit 104 is in a path of an imaging beam 120 emanating from x-ray source 122, such that the beam 120 impinges on the image receptor 116.

The immobilizer unit 104 is supported on a first support arm 124 and the x-ray source 122 is supported on a second support arm 126. For mammography, support arms 124 and 126 can rotate as a unit about an axis 128 between different imaging orientations such as CC and MLO, so that the system 100 can take a mammogram projection image at each orientation. In operation, the image receptor 116 remains in place relative to the platform 106 while an image is taken. The immobilizer unit 104 releases the breast 102 for movement of arms 124, 126 to a different imaging orientation. For tomosynthesis, the support arm 124 stays in place, with the breast 102 immobilized and remaining in place, while at least the second support arm 126 rotates the x-ray source 122 relative to the immobilizer unit 104 and the compressed breast 102 about the axis 128. The system 100 takes plural tomosynthesis projection images of the breast 102 at respective angles of the beam 120 relative to the breast 102. The imaging system may include an acquisition workstation or a technologist workstation which may control the acquisition of the images and may include a display and a user interface for reviewing the images by a technologist. The acquisition workstation may further include a networked computing system that may be connected to a communication network. A technologist operating the imaging system may review any acquired images on the display. In addition, the computing system may receive and process the acquired images. Alternatively, the acquired images may be transmitted via the network to another computing system for processing. The acquisition system may then receive and display the results of processing, such as alerts, indicators, or signals.

Concurrently and optionally, the image receptor 116 may be tilted relative to the breast support platform 106 and in sync with the rotation of the second support arm 126. The tilting can be through the same angle as the rotation of the x-ray source 122, but may also be through a different angle selected such that the beam 120 remains substantially in the same position on the image receptor 116 for each of the plural images. The tilting can be about an axis 130, which can but need not be in the image plane of the image receptor 116. The tilting mechanism 118 that is coupled to the image receptor 116 can drive the image receptor 116 in a tilting motion. For tomosynthesis imaging and/or CT imaging, the breast support platform 106 can be horizontal or can be at an angle to the horizontal, e.g., at an orientation similar to that for conventional MLO imaging in mammography. The system 100 can be solely a mammography system, a CT system, or solely a tomosynthesis system, or a “combo” system that can perform multiple forms of imaging. An example of such a combo system has been offered by the assignee hereof under the trade name Selenia Dimensions.

When the system is operated, the image receptor 116 produces imaging information in response to illumination by the imaging beam 120, and supplies it to an image processor 132 for processing and generating breast x-ray images. A system control and work station unit 138, including software, controls the operation of the system and interacts with the operator to receive commands and deliver information including processed-ray images.

Images may be acquired as a plurality of projections at different angles and thicknesses. The data associated with the plurality of projection images may be processed or reconstructed to produce a plurality of reconstructed images or “slices.” This reconstruction may generally be performed immediately following the image capture sequence. The plurality of reconstructed image slices (or the data associated with the projection images) may be synthesized into a single synthesized image showing the most relevant clinical information and locations of objects of interest. Individual pixels in the final synthesized image may be mapped to a particular image slice. Images may be stored in the data store and can be retrieved by a radiologist for review. The images are then presented to a radiologist who reidentifies objects of interest in the breast that may require additional analysis to determine if the identified objects are potentially cancerous or require a biopsy or monitoring.

One challenge with the imaging system 100 is how to immobilize or compress the breast 102 for the desired or required imaging. A health professional, typically an x-ray technologist, generally adjusts the breast 102 within the immobilizer unit 104 while pulling tissue towards imaging area and moving the compression paddle 108 toward the breast support platform 106 to immobilize the breast 102 and keep it in place, with as much of the breast tissue as practicable being between the compression surfaces 110, 112. This can cause discomfort to the patient, which may be exacerbated by factors such as the length of time of the sweep of the x-ray source to complete the imaging sequence.

Such discomfort is a frequent cause of patient movement during the imaging sequence, resulting in potentially distorted or unusable images. By rapidly identifying images that are distorted or may be distorted, the usability of the images can be assessed immediately. If sequence needs to be reperformed, with may be performed immediately, before the patient leaves the imaging facility.

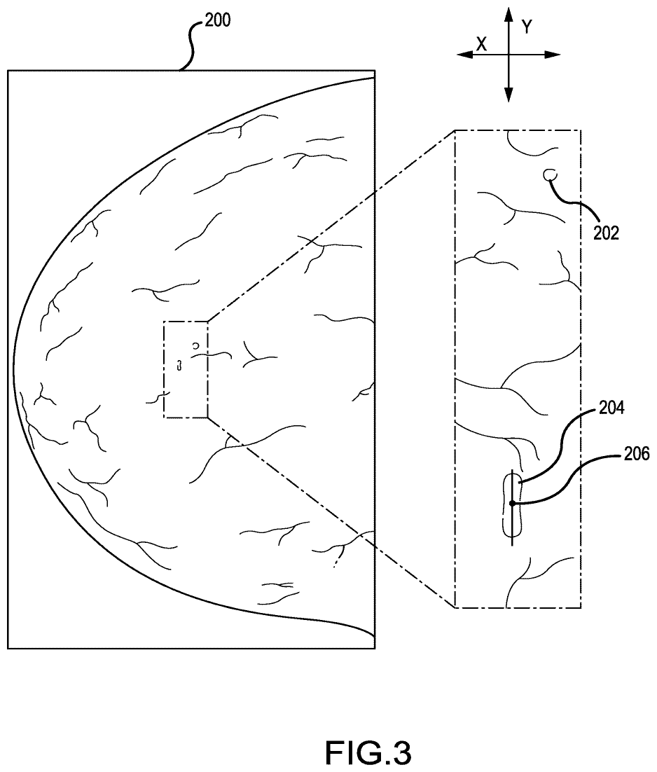

FIG. 3 is an example of an artifact 204 in an image 200. The image 200 may be obtained on the imaging system 100. Example image 200 may be a reconstructed image slice from a tomosynthesis imaging sequence. In examples, image 200 may be an image taken of a patient's breast tissue. Image 200 may be a synthesized or reconstructed image output by image processing of tomosynthesis projection images.

Image 200 also contains an example high contrast object 202. High contrast object 202 appears in focus in the example image 200. A high contrast object is an object appearing against the background of the patient's tissue with a relatively high contrast, as compared to the background tissue and other objects within the image. While the total contrast of an image refers to the spectrum of brightness of the elements within the image, high contrast, as used herein, refers to an image containing elements of high brightness adjacent to elements with low brightness. In comparison, an image characterized as medium contrast would have a wide range of tones with small changes in brightness between adjacent elements.

Brightness may be determined according to pixel values associated with a particular element of an image, e.g., an object, as compared with pixel values associated with an adjacent element of the image, e.g., a background. Contrast may be understood as a degree of difference between the pixel values associated with the object and the pixel values associated with the background. High contrast may be understood as the degree of difference meeting or exceeding a predetermined threshold. For example, many computer color palettes contain pixel values ranging from 0 (black) to a maximum (white). The predetermined threshold may be a fixed value or a percentage of the overall range. In examples, the predetermined threshold may also include a predetermined distance between contrasting pixels. In examples, contrast may be evaluated based upon a histogram and calculating a distance between a maximum and a minimum pixel value. In examples, a contrast analysis may be limited to a portion of an image, such as a portion of the image nearest to a candidate high contrast object.

A high contrast object is generally a relatively denser object within the tissue of the breast. In examples, a high contrast object may be formed in an image due to the presence of a naturally occurring object in the breast, such as a calcification, a ligament, etc., within the breast. A high contrast object may be formed in an image due to the presence of an artificial objects within the breast, such as metallic clips or wires that may be implanted following a biopsy or a similar procedure. Object 202 may appear in other image slices, where it is not in focus, as an artifact.

Artifact 204 may represent a different high contrast object, other than object 202, which is out of focus in image 200. Artifact 204 may be a shadow cast by a high contrast object located elsewhere in the breast. For example, as discussed above, in tomosynthesis a number of images are taken at different projection angles and these images are then processed to reconstruct the image slices, such as image 200. Thus, objects in other areas of the breast, which are out of focus in a particular slice, may still cast shadows and thus produce artifacts on that particular slice, due to the projection angle.

Artifact 204 appears as an unfocused high contrast object that extends linearly in the direction of the motion of the x-ray source (the y-direction in FIG. 3). This extension of the artifact is a function of the angle of projection, which in some cases produces differing resolutions in some directions. For example, artifact 204 may result from the particular angle of projection have a relatively lower resolution in a z-direction, as compared to the x- and y-directions. The length of these artifacts can therefore vary with the imaging modality. For example, conventional tomosynthesis uses a 15 degree sweep and may produce artifacts such as artifact 204, while imaging modalities with wider angles, e.g., at 20 degrees, 30 degrees, 40 degrees, 50 degrees, 60 degrees, etc., may produce artifacts with a smaller, shorter, or otherwise reduced appearance as compared to the artifacts appearing at 15 degrees. Imaging systems with projection angles of 180 degrees or more may eliminate such artifacts entirely.

In an example, a physical attribute of artifact 204 can be identified by evaluating an extension of artifact 204 away from a center point 206 in each of an x- and y-direction. Artifact 204 appears as a straight line extending away from center point 206 in the direction of motion of the x-ray source (the y-direction in FIG. 3). The artifact appearing as a straight line, e.g., no deviation of the artifact in the x-direction of FIG. 3, is consistent with baseline characteristics and indicates a lack of movement of the patient during the image capture.

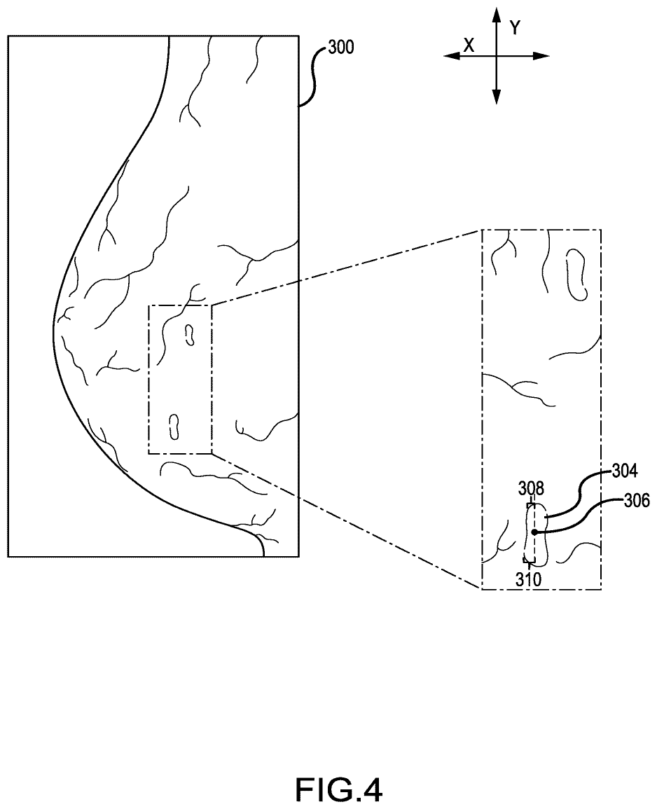

FIG. 4 is an example image 300 of an artifact 304 in an instance where patient motion is detected. Example image 300 may be a reconstructed image slice from a tomosynthesis imaging sequence. In examples, image 300 may be an image taken of a patient's breast tissue. Image 300 may be a synthesized or reconstructed image output by image processing of tomosynthesis image slices.

Artifact 304, as compared to artifact 204 of FIG. 3, shows marked deviation 308, 310 in an x-direction, indicating the image capture of this artifact was influenced by motion other than the motion of the x-ray source. Since movement of the x-ray source is in the y-direction, as identified on FIG. 4, any distortion of the artifact 304 away from a line parallel to the y-axis indicates movement of the image subject, e.g., the patient or the imaged portion of the patient. The present disclosure provides systems and methods for performing a comparison between one or more measured attributes associated with an identified artifact and one or more baseline characteristics, and making a determination of whether patient movement occurred based on the comparison. Further, the present disclosure provides systems and methods for determining whether any movement has exceeded a threshold beyond which the resulting images may be diagnostically invalid.

In examples, deviation may be measured as a number of degrees, a number of pixels, a unit of length (e.g., millimeters) measuring a width of the artifact, a center point of the artifact, a margin of the artifact, an opacity of the artifact, etc. Deviation may be evaluated based on a level of contrast between the artifact and a background, or a comparison to an associated high contrast object, e.g., the high contrast object casting a shadow resulting in the artifact.

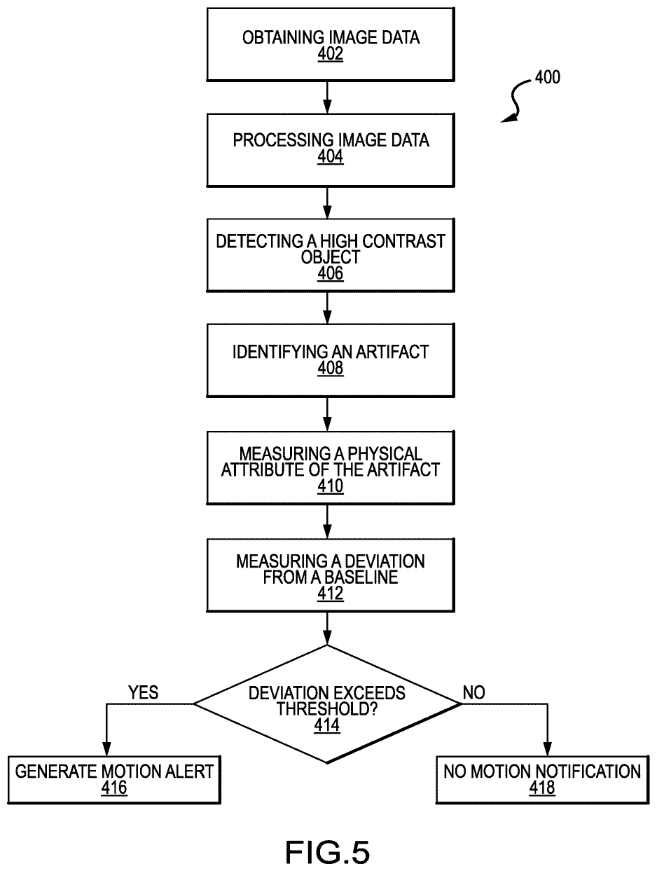

FIG. 5 illustrates an example workflow or method 400 for detecting motion in an image based on comparison of a detected artifact to an artifact baseline. Workflow 400 may be performed by a single integrated system executing one or more models or algorithms, or may be performed by a distributed system providing communication between discrete modules. For example, the workflow may be performed on the imaging system 100.

At 402, image data is obtained. Image data may be obtained using a breast imaging system, for example by mammography, tomosynthesis, or a system combining mammography and tomosynthesis. Obtaining the image data may include emitting an x-ray energy from an x-ray source. The x-ray energy is emitted towards a breast that is immobilized or compressed by a flexible or rigid paddle. Examples of flexible paddles may include those manufactured in part utilizing foam compressive element(s), air filled bladders, etc. The x-ray energy is emitted over a predetermined period of time, e.g., less than about 0.5 sec, about 0.4 sec, about 0.3 sec, and passes through the paddle, breast, etc., and is received at the detector, where the x-ray energy is detected. From this x-ray energy, an x-ray image may be generated. The x-ray image includes at least the imaged breast, including objects within the breast and artifacts resulting from those objects and the motion of the tube or patient. Images may be acquired as a plurality of projections at different angles and thicknesses.

At 404, the image data is processed. The plurality of images may be processed or reconstructed to produce a plurality of reconstructed images or “slices.” This reconstruction may generally be performed immediately following the image capture sequence.

At 406, a high contrast object is detected, such as high contrast object 202 of FIG. 3. Brightness and contrast may be evaluated according to pixel values associated with an object, as compared with pixel values associated with an overall background. The background pixel value, or range of values, may determined to be the most common pixel value among all pixels of the image. Objects may be identified as image elements deviating from the background pixel value by a predetermined number of pixel values or percentage of the overall pixel range. High contrast object may be identified as objects deviating form the background pixel value by a greater number of pixel values or a greater percentage of the overall range. In an example pixel range from 0 (black) to 255 (white), the background pixel values may range from 0-50, objects may be identified as elements defined by pixels with values greater than 160, and high contrast objects as elements defined by pixels with values greater than 200.

In examples, contrast may be evaluated based upon a histogram and calculating a distance between a maximum and a minimum pixel value. In examples, a contrast analysis may be limited to a portion of an image, such as a portion of the image nearest to a candidate high contrast object.

Following, or as part of, the reconstruction, the reconstructed image slices are scanned for the presence of one or more high contrast objects, such as with the use of an image processing algorithm. Some non-limiting examples of image processing algorithms include computer aided detection (CAD) algorithm, a neural network or deep neural network back image processing algorithm, contrast enhancement algorithms, difference-map algorithms, feature and edge detection algorithms, or segmentation algorithms. In examples, one or more physical attributes of the high contrast object may be cataloged and stored to identify the high contrast object and distinguish it from one or more other high contrast objects within the image data. Physical attributes may include the size of the high contrast object, such as length, width, circumference, or diameter, a center point of the high contrast object, a brightness or sharpness of the high contrast object, an opacity of the high contrast object, etc. In some cases, no high contrast objects may be located. If no high contrast objects are present in the image set, an alert or indicator indicating that movement cannot be evaluated due to insufficient presence of high contrast objects may be generated. In such systems, breast movement may be detected via other motion-detection systems, such as the motion detection systems described above.

At 408, an artifact is identified, such as artifact 204 of FIG. 3 or artifact 304 of FIG. 4. The artifact may be identified in response to detecting the high contrast object. For example, once the high contrast object is located in a particular image slice, that may trigger a scan of images one or more slices (such as slices in a tomosynthesis stack of images) away from the high contrast object to find the artifact associated with the high contrast object, e.g., a shadow cast by the high contrast object. In examples, the artifact may be identified independently of the high contrast object.

At 410, a physical attribute of the artifact is measured. The physical attribute may be a size, a shape, a length, a width, a center point, a brightness, a sharpness, etc. For example, each of artifact 204 of FIG. 3 and artifact 304 of FIG. 4 may each have a center point 206, 306 and be measured in the direction of tube movement (a y-direction) and perpendicular to the tube movement (an x-direction) based on the determined center point. In examples, one or more physical attributes of the artifact are measured. In examples, the physical attributes of the artifact may be measured to coincide with the physical attributes of the high contrast object measured, e.g., same attributes, same units, etc.

At 412, the physical attribute of the artifact is compared to a baseline characteristic and a deviation is measured. The baseline characteristic may generally be determined according to a known data set. For example, one or more images may be identified by a radiologist or other clinician as containing an artifact with no patient movement. Artifact 204 of FIG. 3 may represent one artifact identified as suitable for inclusion in a data set for determining the baseline characteristic. The one or more images may be used to form a training set to determine a baseline shape, size, etc. of an artifact with no patient movement. A baseline value or range for one or more baseline characteristics may be determined from the training set.

In examples, determining the baseline may include performing an R-square or another fit analysis to determine a baseline range, e.g., a range of deviation which occurs among the artifacts and does not indicate patient movement. Some differences between artifacts may exist that do not indicate patient movement, such as due to high contrast objects of different sizes and different orientations producing artifacts that likewise have different sizes and orientations. Thus, in examples, the baseline characteristics may incorporate a range in order to encompass differences from a straight line in the direction of motion of the x-ray source.

The baseline characteristics may be determined according to a relationship between a physical attribute of the artifact and a physical attribute of the corresponding high contrast object. For example, a measure of difference in width between the high contrast object and the artifact.

Once determined, the baseline characteristics may be stored and used as a reference for comparison. A detected artifact is compared to the baseline and the deviation of the detected artifact from the baseline is measured. The deviation may be measured using an R-square or another fit analysis to determine a measure of deviation from the baseline by the detected artifact. Measuring the deviation from the baseline may include applying a fitting algorithm. The fitting algorithm may accept as input and consider one or more of a size of the artifact, a size of the associated high contrast object, and a contrast of the artifact.

At 414, the deviation is evaluated against a predetermined threshold. The predetermined threshold may be determined according to the baseline and one or more alert or indicator training images. For example, in addition to the baseline determination discussed above, a threshold determination may also be performed. The threshold determination may include evaluation of one or more training images with artifacts identified as indicating patient motion. There may accordingly be identified both a baseline range, or an amount of deviation at or below which indicates no patient motion, and a threshold deviation, at which or above which indicates patient motion.

In examples, the threshold deviation may be set to a value directly adjacent to the baseline range, such that all images evaluated are determined to either have patient movement or no patient movement. In examples, the threshold deviation may be offset from the baseline range. For example, one or more deviation values may exist between the baseline range and the threshold deviation such that an image may be evaluated to have movement, to have no movement, or to be indeterminate.

In examples, the predetermined threshold may be dynamic and based on feedback from radiologists or other technicians evaluating the image data. For example, the workflow may further include receiving one or more indications of a false positive. One or both of the baseline range and the threshold deviation may be adjusted according to the feedback to require a greater deviation to trigger a determination that the image indicates patient motion.

For example, each of artifact 204 of FIG. 3 and artifact 304 of FIG. 4 may each have a center point 206, 306 and be measured in the direction of tube movement (a y-direction) and perpendicular to the tube movement (an x-direction) based on the determined center point. In examples, measurement in the y-direction may be used to determine two or more locations at which to take measurements in the x-direction. Taking two, or more, measurements in the x-direction on artifact 204 reveals uniform distance from center point 206 along the length of artifact 204, falling within the baseline range and indicating no movement. Taking two, or more, measurements in the x-direction on artifact 304 reveals deviations 308, 310, indicating movement. In examples, either or both of deviations 308, 310 may be compared with the threshold deviation. A difference may be taken between deviations 308, 310, and the difference may be compared to the threshold deviation. At 416, if the deviation exceeds the predetermined threshold, a motion indicator is generated. In examples, the motion indicator may be generated in conjunction with image reconstruction, such that the radiologist or technician is alerted to evaluate the image for useability immediately. In examples, the motion indicator may be a flag or other indicator on the image, or a visual, auditory, haptic, etc. alert at the technician or operator's panel. The motion indicator may be displayed to a technologist on a display. The technologist may evaluate the images acquired and determine if a retake is needed. In other cases, the motion indicator may be stored with the generated image(s) and can be viewed or processed at a later time. In other cases, the motion indicator may interrupt the imaging sequence and indicate to the technologist to initiate a retake. In yet other instances, the imaging system can determine whether to remove images exhibiting excessive motion.

At 418, if the deviation does not exceed the predetermined threshold, an indication of no motion may be generated. In examples, an indicator of no motion may be a visual or auditory indicator that the image is acceptable. The indicator of no motion may include the system processing and storing the image without an overt alert to the operator. In these instances, the indicator that no motion has occurred may be stored associated with the image(s) to be viewed or processed at a later time.

In examples, workflow 400 may be executed as one or more algorithms or modules. For example, a first algorithm may be executed to detect a high contrast object. A second algorithm, which may be informed by the first algorithm, identifies an artifact associated with the high contrast object. Execution of a third algorithm may be performed to evaluate the deviation of the artifact, as compared to a predetermined threshold, and generate an appropriate alert.

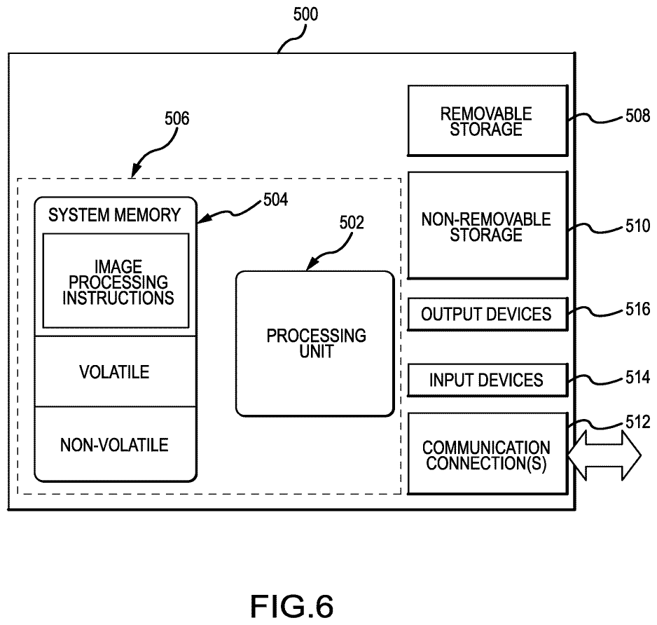

FIG. 6 illustrates one example of a suitable operating environment 500 in which one or more of the present examples can be implemented. This operating environment may be incorporated directly into the imaging systems disclosed herein, or may be incorporated into a computer system discrete from, but used to control, the imaging and compression systems described herein. This is only one example of a suitable operating environment and is not intended to suggest any limitation as to the scope of use or functionality. Other well-known computing systems, environments, and/or configurations that can be suitable for use include, but are not limited to, imaging systems, personal computers, server computers, hand-held or laptop devices, multiprocessor systems, microprocessor-based systems, programmable consumer electronics such as smart phones, network PCs, minicomputers, mainframe computers, tablets, distributed computing environments that include any of the above systems or devices, and the like.

In its most basic configuration, operating environment 500 typically includes at least one processing unit 502 and memory 504. Depending on the exact configuration and type of computing device, memory 504 (storing, among other things, instructions to identify high contrast objects and artifacts, to associate artifacts with corresponding high contrast objects and vice versa, to measure one or more attributes of a high contrast object and/or an artifact, to determine baseline characteristics of artifacts, to measure a deviation of an artifact, or perform other methods disclosed herein) can be volatile (such as RAM), non-volatile (such as ROM, flash memory, etc.), or some combination of the two. This most basic configuration is illustrated in FIG. 6 by dashed line 506. Further, environment 500 can also include storage devices (removable, 508, and/or non-removable, 510) including, but not limited to, magnetic or optical disks or tape. Similarly, environment 500 can also have input device(s) 514 such as touch screens, keyboard, mouse, pen, voice input, etc., and/or output device(s) 516 such as a display, speakers, printer, etc. Also included in the environment can be one or more communication connections 512, such as LAN, WAN, point to point, Bluetooth, RF, etc.

Operating environment 500 typically includes at least some form of computer readable media. Computer readable media can be any available media that can be accessed by processing unit 502 or other devices having the operating environment. By way of example, and not limitation, computer readable media can include computer storage media and communication media. Computer storage media includes volatile and nonvolatile, removable and non-removable media implemented in any method or technology for storage of information such as computer readable instructions, data structures, program modules or other data. Computer storage media includes, RAM, ROM, EEPROM, flash memory or other memory technology, CD-ROM, digital versatile disks (DVD) or other optical storage, magnetic cassettes, magnetic tape, magnetic disk storage or other magnetic storage devices, solid state storage, or any other tangible medium which can be used to store the desired information. Communication media embodies computer readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media. A computer-readable device is a hardware device incorporating computer storage media.

The operating environment 500 can be a single computer operating in a networked environment using logical connections to one or more remote computers. The remote computer can be a personal computer, a server, a router, a network PC, a peer device or other common network node, and typically includes many or all of the elements described above as well as others not so mentioned. The logical connections can include any method supported by available communications media. Such networking environments are commonplace in offices, enterprise-wide computer networks, intranets and the Internet.

In some examples, the components described herein include such modules or instructions executable by computer system 500 that can be stored on computer storage medium and other tangible mediums and transmitted in communication media. Computer storage media includes volatile and non-volatile, removable and non-removable media implemented in any method or technology for storage of information such as computer readable instructions, data structures, program modules, or other data. Combinations of any of the above should also be included within the scope of readable media. In some examples, computer system 500 is part of a network that stores data in remote storage media for use by the computer system 500.

In examples, the various systems and methods disclosed herein may be performed by one or more server devices. For example, in one example, a single server may be employed to perform the systems and methods disclosed herein, such as the methods for imaging discussed herein. Client device 502 may interact with a server via network. In further examples, the client device 502 may also perform functionality disclosed herein, such as scanning and image processing, which can then be provided to a server or servers.

### EXAMPLES

Illustrative examples of the systems and methods described herein are provided below. An embodiment of the system or method described herein may include any one or more, and any combination of, the clauses described below.

Clause 1. A method of detecting motion of a patient's breast tissue during medical imaging, the method comprising: obtaining image data of the patient's breast tissue; processing the image data to generate a set of image slices that collectively depict the patient's breast tissue; detecting a high contrast object on one or more image slices of the set of image slices; identifying an artifact, associated with the high contrast object, on one or more image slices of the set of image slices; measuring a physical attribute of the artifact; measuring a deviation of the physical attribute from a baseline; determining the deviation exceeds a predetermined threshold; and generating, in response to determining the deviation exceeds the predetermined threshold, a motion indicator.

Clause 2. The method of clause 1, wherein the baseline is a straight line.

Clause 3. The method of clause 1 or 2, wherein the baseline further comprises a baseline range.

Clause 4. The method of any of clauses 1-3, wherein the baseline range is determined according to a set of physical attributes associated with one or more artifacts.

Clause 5. The method of clause 4, wherein the one or more artifacts are associated with no patient movement.

Clause 6. The method of any of clauses 1-5, wherein the predetermined threshold is determined according to the baseline range.

Clause 7. The method of any of clauses 1-6, wherein the baseline range is measured using an R-square analysis.

Clause 8. The method of any of clauses 1-7, wherein the high contrast object is identified on an in-focus image slice and the artifact is a projection of the high contrast object identified on an out-of-focus image slice.

Clause 9. The method of any of clauses 1-8, wherein detecting a high contrast object comprises identifying at least one high contrast object in a first image slice and, in response, scanning a second image slice one or more slices away from the first image slice for the artifact.

Clause 10. The method of any of clauses 1-9, wherein the physical attribute includes one or more of a width, a length, a height, a trajectory, and an opacity.

Clause 11. The method of clause 10, wherein determining the deviation exceeds a predetermined threshold is based on measuring the physical attribute associated with the artifact and measuring the at least one high contrast object.

Clause 12. The method of any of clauses 1-11, wherein measuring the deviation from the baseline comprises applying a fitting algorithm, wherein inputs to the fitting algorithm comprise one or more of a size of the artifact, a size of the associated high contrast object, and a contrast of the artifact.

Clause 13. The method of any of clauses 1-12, wherein the high contrast object is a naturally occurring object in the breast including one or more of a calcification or a ligament.

Clause 14. The method of any of clauses 1-13, wherein the high contrast object is an implanted object.

Clause 15. The method of any of clauses 1-14, wherein the motion indicator comprises an indicator on at least one image of the one or more image slices.

Clause 16. The method of any of clauses 1-15, wherein the motion indicator comprises a request for immediate review of at least one image of the one or more images.

Clause 17. The method of any of clauses 1-16, wherein measuring the deviation from the baseline associated with the artifact comprises using an R-square analysis.

Clause 18. A system comprising: a computer-readable memory storing executable instructions; and one or more processors in communication with the computer-readable memory, wherein, when the one or more processors execute the executable instructions, the one or more processors perform: obtaining image data of the patient's breast tissue; processing the image data to generate a set of image slices that collectively depict the patient's breast tissue; detecting a high contrast object on one or more image slices of the set of image slices; identifying an artifact, associated with the high contrast object, on one or more image slices of the set of image slices; measuring a physical attribute of the artifact; measuring a deviation of the physical attribute from a baseline; determining the deviation exceeds a predetermined threshold; and generating, in response to determining the deviation exceeds the predetermined threshold, a motion indicator.

Clause 19. A non-transitory computer readable medium having stored thereon one or more sequences of instructions for causing one or more processors to perform: obtaining image data of the patient's breast tissue; processing the image data to generate a set of image slices that collectively depict the patient's breast tissue; detecting a high contrast object on one or more image slices of the set of image slices; identifying an artifact, associated with the high contrast object, on one or more image slices of the set of image slices; measuring a physical attribute of the artifact; measuring a deviation of the physical attribute from a baseline; determining the deviation exceeds a predetermined threshold; and generating, in response to determining the deviation exceeds the predetermined threshold, a motion indicator.

This disclosure described some examples of the present technology with reference to the accompanying drawings, in which only some of the possible examples were shown. Other aspects can, however, be embodied in many different forms and should not be construed as limited to the examples set forth herein. Rather, these examples were provided so that this disclosure was thorough and complete and fully conveyed the scope of the possible examples to those skilled in the art.

Although various embodiments and examples are described herein, those of ordinary skill in the art will understand that many modifications may be made thereto within the scope of the present disclosure. Therefore, the specific structure, acts, or media are disclosed only as illustrative examples. Examples according to the technology may also combine elements or components of those that are disclosed in general but not expressly exemplified in combination, unless otherwise stated herein. Accordingly, it is not intended that the scope of the disclosure in any way be limited by the examples provided.

1. A method of detecting motion of a patient's breast tissue during medical imaging, the method comprising:
obtaining image data of the patient's breast tissue;
processing the image data to generate a set of image slices that collectively depict the patient's breast tissue;
detecting a high contrast object on one or more image slices of the set of image slices;
identifying an artifact, associated with the high contrast object, on one or more image slices of the set of image slices, wherein the artifact is a projection of the high contrast object identified on an out-of-focus image slice;
measuring a physical attribute of the artifact, wherein the physical attribute includes one or more of a width, a length, a height, and an opacity;
measuring a deviation of the physical attribute from a baseline;
determining the deviation exceeds a predetermined threshold; and
generating, in response to determining the deviation exceeds the predetermined threshold, a motion indicator.

2. The method of claim 1, wherein the baseline is a straight line.

3. The method of claim 1, wherein the baseline further comprises a baseline range.

4. The method of claim 1, wherein the baseline range is determined according to a set of physical attributes associated with one or more artifacts.

5. The method of claim 4, wherein the one or more artifacts are associated with no patient movement.

6. The method of claim 1, wherein the predetermined threshold is determined according to the baseline range.

7. The method of claim 1, wherein the baseline range is measured using an R-square analysis.

8. The method of claim 1, wherein the high contrast object is identified on an in-focus image slice.

9. The method of claim 1, wherein detecting a high contrast object comprises identifying at least one high contrast object in a first image slice and, in response, scanning a second image slice one or more slices away from the first image slice for the artifact.

10. (canceled)

11. The method of claim 1, wherein determining the deviation exceeds a predetermined threshold is based on measuring the physical attribute associated with the artifact and measuring the at least one high contrast object.

12. The method of claim 1, wherein measuring the deviation from the baseline comprises applying a fitting algorithm, wherein inputs to the fitting algorithm comprise one or more of a size of the artifact, a size of the associated high contrast object, and a contrast of the artifact.

13. The method of claim 1, wherein the high contrast object is a naturally occurring object in the breast including one or more of a calcification or a ligament.

14. The method of claim 1, wherein the high contrast object is an implanted object.

15. The method of claim 1, wherein the motion indicator comprises an indicator on at least one image of the one or more image slices.

16. The method of claim 1, wherein the motion indicator comprises a request for immediate review of at least one image of the one or more images.

17. The method of claim 1, wherein measuring the deviation from the baseline associated with the artifact comprises using an R-square analysis.

18. A system comprising:
a computer-readable memory storing executable instructions; and
one or more processors in communication with the computer-readable memory, wherein, when the one or more processors execute the executable instructions, the one or more processors perform:
obtaining image data of the patient's breast tissue;
processing the image data to generate a set of image slices that collectively depict the patient's breast tissue;
detecting a high contrast object on one or more image slices of the set of image slices;
identifying an artifact, associated with the high contrast object, on one or more image slices of the set of image slices, wherein the artifact is a projection of the high contrast object identified on an out-of-focus image slice;
measuring a physical attribute of the artifact, wherein the physical attribute includes one or more of a width, a length, a height, and an opacity;
measuring a deviation of the physical attribute from a baseline;
determining the deviation exceeds a predetermined threshold; and
generating, in response to determining the deviation exceeds the predetermined threshold, a motion indicator.

19. A non-transitory computer readable medium having stored thereon one or more sequences of instructions for causing one or more processors to perform:
obtaining image data of the patient's breast tissue;
processing the image data to generate a set of image slices that collectively depict the patient's breast tissue;
detecting a high contrast object on one or more image slices of the set of image slices;
identifying an artifact, associated with the high contrast object, on one or more image slices of the set of image slices, wherein the artifact is a projection of the high contrast object identified on an out-of-focus image slice;
measuring a physical attribute of the artifact, wherein the physical attribute includes one or more of a width, a length, a height, and an opacity;
measuring a deviation of the physical attribute from a baseline;
determining the deviation exceeds a predetermined threshold; and
generating, in response to determining the deviation exceeds the predetermined threshold, a motion indicator.

20. The method of claim 1, wherein the physical attribute includes a trajectory.

21. The method of claim 1, wherein measuring the physical attribute of the artifact comprises measuring at least one of a center point of the artifact and an outline of the artifact.
